(19) United States
(12) Patent Application Publication
(10) Pub. No.: US 2010/0095957 A1
(43) Pub. Date: Apr. 22, 2010

(54) Manually operated monodose nasal sprayer device

(75) Inventor: Hipolito P. Corbacho, Green Brook, NJ (US)

Correspondence Address: Deirdra M. Meagher, 24 Mine Street, Flemington, NJ, 08822, US

(73) Assignee: Corbco, Inc.

(21) Appl. No.: 12/590,522

(22) Filed: Nov. 10, 2009

Related U.S. Application Data
(63) Continuation-in-part of application No. 12/011,117, filed on Jan. 24, 2008.
Application No. 12/011,117 is a continuation-in-part of application No. 11/713,335, filed on Mar. 2, 2007.

(51) Int. Cl. A61M 15/08

(52) U.S. Cl. 128/200.14

(57) ABSTRACT

The nasal sprayer utilizes a piston that fires automatically upon full depression of the firing cap. The drive spring does not fire the piston until fully compressed because the force required to break a puncturable medicine container is greater than to compress the spring. The piston height is greater than the firing cap and central member height combined so that after the spring is fully compressed, the piston punctures the medicine container. The spring then releases, pushing the piston toward the nozzle. A user procures medicine from the device by (i) unlocking a child resistant lock; (ii) applying compressive force against the firing cap to compress the spring and to push the piston to puncture the medicine container, then the spring causes the distal end of the piston to drive medicine through the spray nozzle independent of the user as to dosage amount and speed of delivery.

### REFERENCE TO RELATED APPLICATIONS

This application is a continuation-in-part of copending U.S. patent application Ser. No. 12/011,117 filed on Jan. 24, 2008, entitled “Manually Operated Monodose Nasal Sprayer” by the same inventor herein, which is itself a continuation-in-part of copending U.S. patent application Ser. No. 11/713,335 filed on Mar. 2, 2007, entitled “Monodose Nasal Sprayer” by the same inventor herein.

### BACKGROUND OF INVENTION

a. Field of Invention

The invention relates generally to nasal sprayers and, more particularly, to nasal sprayers that deliver a single dose that is user independent as to dosage amount and dosage speed of delivery to yield a consistent, predetermined dose and profile spray. The user will activate the internal mechanism by placing the delivery (distal) end of the device to the nasal area and squeezing the two external components (the firing cap and the central member) together to trigger the automatic release.

b. Description of Related Art

The following patents are representative of sprayers:

U.S. Pat. No. 7,000,806 B2 to Daniel Py et al. describes a dispenser for dispensing a fluid which includes a rigid vial that has a main fluid chamber containing a fluid, and a pump assembly that is in fluid communication with the main fluid chamber and is configured to dispense a predetermined quantity of fluid from the main fluid chamber. A flexible bladder is provided which is located within the main fluid chamber and is configured to expand to fill the ullage created within the main fluid chamber during dispensing of fluid by the pump assembly. The resilient bladder tends to force itself outwardly toward the rigid vial and, in turn, increases the pressure within the main fluid chamber in comparison to the interior of the bladder to thereby prevent the ingress of air or vapors through the bladder or otherwise into the main fluid chamber.

U.S. Pat. No. 6,997,219 to Daniel Py et al. describes a dispenser for holding multiple doses of fluids or other substances, and for dispensing the substances, which has a vial, a flexible bladder received within the vial, and a variable volume storage chamber formed between the bladder and vial. A filling valve is coupled in fluid communication with the storage chamber and defines a normally closed, fluid-tight position hermetically sealing the storage chamber from the ambient atmosphere, and an open position allowing the passage of fluid through the valve both to evacuate the storage chamber and to introduce fluid through the valve to fill the storage chamber. A pump is coupled in fluid communication with the storage chamber for pumping fluids out of the storage chamber. A dispensing valve is coupled in fluid communication with the pump and defines a normally closed, fluid-tight position preventing the passage of fluid out of the dispenser, and an open position for dispensing pumped fluid therethrough. The sealed, empty dispenser is sterilized, such as by applying gamma radiation thereto. Then, the sterilized, sealed, empty dispenser is filled with fluid by engaging the filling valve with an evacuating/dispensing member to evacuate the storage chamber, and by introducing fluid from the filling member through the open filling valve and into the storage chamber. The filling member is withdrawn from the valve, and a spring moves the valve to a closed position to hermetically seal the fluid within the dispenser.

U.S. Pat. No. 6,419,663 B2 to John E. Harrold describes the present invention mechanically propelled, liquid metered dispenser which includes a main housing with liquid storage and a liquid advancing component for either allowing liquid to flow out or assisting in the flow of liquid out of the main housing by exerting pressure, into a metered dosage dispensing chamber in response to a loading movement of a cocking mechanism. The chamber has an inlet connected to the main housing with a one-way valve to only permit flow of liquid into the chamber. The chamber also has an outlet orifice for dispensing liquid therefrom in a metered amount. The chamber has a reciprocal plunger and has a load and release component connected to it, which includes a cocking mechanism is functionally connected to the plunger so as to move in harmony therewith, or, more preferably, to move a relatively short distance relative to the distance traveled by the cocking mechanism.

U.S. Pat. No. 6,302,101 B1 to Daniel Py describes a pump type dispenser for dispensing predetermined doses of medicament in droplets or in spray form to the nasal area which incorporates a rigid vial for medicament, and expandable pouch located within the rigid vial, a nasal screen, a one-way actuation mechanism, a one-way valve mechanism in the nozzle area and a spring element, both the valve mechanism and the spring element being formed as integral portions of the pump body. The nasal screen aligns the dispenser nozzle with the nasal passage and also allows the user to discreetly hide the nasal area from the public view. The one-way valve mechanism in the nozzle area ensures a one-way movement of medicament from the dispenser, thereby preserving substantially perfect sterility of the medicament in the dispenser regardless of the environment surrounding the dispenser, without requiring the use of preservatives. The one way actuation mechanism enables the user to load and dispense a uniform quantity of medicament with a uniform actuation force and speed via a single continuous motion of the actuation mechanism upon application of a very small force on the actuation trigger mechanism by the user. By preventing the pump mechanism from being left in a loaded state for a prolonged period of time, the one-way actuation mechanism prevents the spring element from experiencing the “fatigue” phenomenon. The rigid vial/expandable-pouch combination facilitates improved long term use, as well as uniformity of dosage independent of the pump orientation.

U.S. Pat. No. 6,033,384 to Daniel Py describes a medicament-dispensing system which includes a cartridge for housing and actuating an accordion-like vial-dispenser for dispensing a calibrated amount of medicament by means of a single actuation motion which sequentially loads the vial-dispenser and dispenses the loaded medicament. The vial-dispenser has a front bellows portion, a rear bellows portion, an internal piston mechanism, a medicament storage chamber and a dosage cavity. The cartridge has a trigger mechanism which acts in concert with a notched lever and a wedge-shaped arm internally located in the cartridge to sequentially load the dosage cavity with medicament and discharge it. Depression of the trigger mechanism simultaneously extends, by means of the notched lever, the front bellows portion and compresses the rear bellows portion to load the dosage cavity with medicament. Once the notched lever has extended the front bellows portion a predetermined distance, the notched lever is disengaged from the front bellows portion by the wedge-shaped arm extending from the rear wall of the housing, thereby releasing the front and rear bellows portions, along with the internal piston mechanism, to return to original position and force the medicament from the dosage cavity via a nozzle of the vial-dispenser.

U.S. Pat. No. 5,979,711 to Karl-Heinz Fuchs et al. describes that in a device suitable for discharging media or at least one springly deformable component consists of a copolymer produced with a co-catalyst such as titanocen, more particularly an ethylene ex-olefine copolymer which may also be improve as regards its resiliency by gamma irradiation. In a spiral spring produced from this or a similar plastics material axially adjacent spiral sections are directly integrally connected to each other not only via the spiral but also along the circumference of the spring.

U.S. Pat. No. 5,685,869 to Daniel Py describes an apparatus that is used to apply medicament to an eye and stores the medicament in a medicament chamber. A nozzle is coupled in fluid communication with the medicament chamber and is formed by an outer nozzle portion and an inner nozzle portion received within the outer nozzle portion. A seam is formed by the interface of the inner nozzle portion and the outer nozzle portion and is normally in a closed position to prevent the passage of medicament through the nozzle. The seam opens in response to the flow of medicament of sufficient pressure into the seam to permit the passage of medicament through the nozzle for release into the eye.

U.S. Pat. No. 5,613,957 to Daniel Py describes an apparatus that is used to apply medicament to an eye and stores the medicament in a medicament chamber. A nozzle is coupled in fluid communication with the medicament chamber and is formed by an outer nozzle portion and an inner nozzle portion received within the outer nozzle portion. A tight interface is defined between the inner nozzle portion and the outer nozzle portion and is normally in a closed position to prevent the passage of medicament through the nozzle. The interface opens in response to the flow of medicament of sufficient pressure into it to permit the passage of medicament through the nozzle for release into the eye.

U.S. Pat. No. 5,401,259 to Daniel Py describes a cartridge for actuating a piston-like or accordion-like dispenser-vial for applying a medicament to an eye. The cartridge includes a housing for holding the dispenser-vial and a telescoping cylinder for compressing the dispenser-vial in the longitudinal direction to actuate the vial. The cartridge includes a locking mechanism for locking the telescoping cylinder to restrict its movement and a lever mechanism for releasing the cylinder from the locked position so that a drop is released from the dispenser. The housing includes a finger for engaging the lower eyelid and exposing the conjunctival cul-de-sac.

U.S. Pat. No. 5,267,986 to Daniel Py describes a cartridge for actuating a piston-like or accordion-like dispenser-vial for applying medicament to an eye. The cartridge includes a housing for holding the dispenser-vial and a telescoping cylinder for compressing the dispenser-vial in the longitudinal direction to actuate the vial. The cartridge includes a locking mechanism for locking the telescoping cylinder to restrict its movement and a trigger mechanism for releasing the cylinder from the locked position so that a drop is released from the dispenser. The housing includes a finger for engaging the lower eyelid and exposing the conjunctival cul-de sac.

U.S. Pat. No. 5,238,156 to Raimund Andris describes that in a metering and spray pump for liquid, low-viscosity and pasty substances, an elastic bellows is arranged between two plastic housing parts that are telescopingly movable relative to one another, connecting them. The bellows, acting as a discharge valve has, at one end, a valve annular wall that surrounds the generated surface of an inner annular discharge seat made in one piece with the first housing part in a sealing manner and such that it can be lifted off. As a suction valve the bellows has, at its other end, a valve annular wall which is in sealing and separable contact with the generated surface of a valve seat made in one piece with the second housing part such that it can be lifted off, and the medium to be pumped is drawn into the bellows through the valve seat. To guarantee high reliability of operation, especially good closing quality at weak valve opening forces, where the quality of closing can be tested even in the dry state, with the smallest possible number of simple and easy to assemble individual parts, the valve annular wall of the discharge valve and the valve annular wall of the suction valve, which valve annular wall is provided with a closed front wall, are each is contact with conical or hemispherical generated surfaces, wherein both valve annular walls are connected to the bellows both radially elastically and elastically movable in the axial direction.

U.S. Pat. No. 4,944,430 to Lothar Graf et al. describes a vacuum-tight and pressure-tight closed storage space in a container that is constructionally combined with a thrust piston pump projecting thereinto and a drag piston slideably guided therein and in the initial position of the thrust piston pump is hermetically sealed by an exhaust valve mechanically closed in forcibly controlled manner. The drag piston is adapted to the thrust piston pump in such a way that the storage space can be emptied substantially free with the thrust piston pump.

U.S. Pat. No. 3,934,585 to David M. Maurice describes a method and apparatus for applying therapeutic eye drops to the eye by metering a predetermined volume of fluid and rapidly applying a pressure to one end of the metered fluid for forcing the fluid from a nozzle of means defining a small passageway such as an open ended tube as a droplet having sufficient velocity to travel a generally horizontal distance in space to the eye. Unit dose application and multiple dose application are included and provision is made for preventing anticipatory blinking of the eye during self-administration.

Notwithstanding the prior art, the present invention is neither taught nor rendered obvious thereby.

### SUMMARY OF INVENTION

The present invention is a monodose nasal sprayer device that eliminates the usual uncertainties of nasal sprayers, to wit, the speed of depression that affects the size and shape (profile) of the spray which in turn affects the efficacy of delivery; and the length of depression of the a typical nasal spray pump mechanism that affects the amount of dosage. These variables can have adverse affects on the proper treatment of a nasal condition and are completely eliminated by the present invention. Thus, the present invention has been developed to drive medicine through the spray nozzle in a manner that is user independent as to dosage amount and dosage speed of delivery to yield a consistent, predetermined dosage and a consistent predetermined profile spray.

The present invention includes an elongated main housing, a central member and a firing cap, as well as internal components. The main housing has a distal end adapted for partial insertion into one of a human nasal cavity and an animal nasal cavity and has a proximal end adapted to receive and hold the breakable monodose medicine package-supporting central member. The elongated main housing has a spray discharge nozzle located at its distal end, and it has a medicine-receiving chamber connected to the nozzle and biased toward the breakable monodose medicine package-supporting central member.

The central member is a breakable medicine package-supporting central member that is connected to the proximal end of the elongated main housing. The connection may be permanent or removable, and it may be threaded, heat welded, snap fitted or otherwise connected and the central unit may be connect into the inside of the main housing, or vice versa. The central member has a distal end adapted to support a breakable monodose medicine package so as to be positioned at the chamber, and has a piston shaft with a piston-supporting area open at the distal end and adapted to extend a piston therein. A piston is partially located in the piston shaft, the piston having a medicine container breaking distal end, and has a downwardly extended proximal end functionally connected to a firing cap. A firing cap is connected to the proximal end of the central member and is moveable toward the distal end of the central member by compressive force. The firing cap has a piston-receiving cavity and a drive spring located in the cavity and the extended proximal end of the piston is functionally connected to the spring. A finger support means is located on the outside of one of the main housing and the central member. A child resistant inner locking mechanism is connected to at least one of the central member and the firing cap that is unlockable and, when locked, prevents movement of the firing cap toward the distal end of the central member, and permits movement of the firing cap toward the distal end of the central member when the locking member is unlocked. A child resistant outer locking mechanism is connected to the firing cap and the central member, and is adapted to prevent movement of the firing cap. The child resistant outer locking mechanism is a tear away system including a connection means for connecting said firing cap to said central member. The outer locking mechanism connection means includes a perforated tab surrounding a circumference of at least one of said firing cap and said central member. The piston has a predetermined height, the central member pistol-supporting area has a predetermined height, and the firing cap has a predetermined height, and wherein the height of the piston is greater than the sum of the height of the central member piston-supporting area and the firing cap.

An essential feature is that the force required to overcome the frictional force between the high friction engaging surface of the piston and the piston shaft is greater than the force required to compress the spring. A user may procure a monodose of medicine with the device when it is loaded with a breakable monodose medicine package by (i) removing the child resistant outer locking mechanism so that its position is unlocked; (ii) moving the inner child resistant locking mechanism from its locked position to its unlocked position; (iii) placing fingers in front of the finger support means and applying compressive force against the firing cap to move the firing cap towards the central member so as to first compress the spring before moving the piston, and to move the firing cap closer toward the central member to push the piston out of the piston engaging friction area of the shaft so as to sufficiently reduce friction such that the spring automatically and with significant celerity, fires the piston and causes the distal end of the piston to puncture the breakable monodose medicine package. This automatically drives medicine therefrom to and through the spray nozzle in a manner that is user independent as to dosage amount and dosage speed of delivery to yield a consistent, predetermined speed of delivery and a predetermined profile spray.

In some preferred embodiments, the manually operated monodose nasal sprayer includes a piercer attached to a proximal end of the single use monodose medicine package wherein when the piston is fired the piercer moves upwardly through the single use monodose medicine package to pierce the monodose medicine package and to drive medicine therefrom to and through the nozzle. The single monodose medicine package has side walls, a rupturable top and a rupturable push-in bottom.

In some preferred embodiments, the present invention monodose nasal sprayer device piston shaft and the piston have corresponding circular peripheries from a top view.

In some preferred embodiments of the present invention monodose nasal sprayer device, the distal end of the central member is connected to the proximal end of the main housing through connection means wherein the connection means is selected from one of the group consisting of corresponding screw components and corresponding snap components.

In some preferred embodiments of the present invention monodose nasal sprayer device, the screw components include a one way locking thread stop so that the distal end of the central member and the proximal end of the main housing cannot be unscrewed once screwed together.

In some preferred embodiments of the present invention monodose nasal sprayer device, the proximal end of the piston includes a flange that fits atop the spring and fits into the firing cap cavity.

In some preferred embodiments of the present invention monodose nasal sprayer device, the child resistant locking mechanism is a rotating locking mechanism with a first position being a lock position to inhibit advancement of the firing cap and a second position being an unlock position to permit advancement of the firing cap, the second position being located at an arc of a predetermined distance from the first position.

In some preferred embodiments of the present invention monodose nasal sprayer device, the child resistant locking mechanism includes a protrusion on the firing cap and a receiving track on the central member such that when the protrusion is positioned in alignment with the receiving track, it is in its unlock position and the firing cap may be advanced toward the central member.

In some preferred embodiments of the present invention monodose nasal sprayer device, the device further includes a breakable monodose medicine package mounted on the distal end of the central member and at least partially projecting into the chamber.

In some preferred embodiments of the present invention monodose nasal sprayer device, at least one of the piston and the shaft includes at least one friction ring.

In some preferred embodiments of the present invention monodose nasal sprayer device, the distal end of the central member and the proximal end of the main housing have corresponding snap-together components and the central member distal end and the main housing proximal end are snapped into one another.

In another preferred embodiment of the present invention monodose nasal sprayer device, the device includes: a.) an elongated main housing has a distal end adapted for partial insertion into a one of human nasal cavity and an animal nasal cavity and having a proximal end adapted to receive and hold a monodose medicine package-supporting central member. The elongated main housing has a spray discharge nozzle located at its distal end, the elongated main housing having medicine-receiving chamber connected to said nozzle and biased toward the monodose medicine package-supporting central member; b.) the medicine package-supporting central member being connected to the proximal end of the elongated main housing, the central member has a distal end adapted to support a monodose medicine package so as to be positioned at the chamber, and has a piston shaft with a piston-engaging high friction area end and having a piston-releasing area distal end; c.) a piston located in the piston shaft, the piston has a medicine container breaking distal end, has a central area with a high friction engaging surface to engage the proximal end of the piston shaft and has an extended proximal end functionally connected to a firing cap; d.) a firing cap connected to the proximal end of the central member and being moveable toward the distal end of the central member by compressive force, the firing cap having a piston-receiving cavity and a drive spring located in the cavity, the extended proximal end of the piston being functionally connected to the spring; e.) finger support means is located on the outside of one of the main housing and the central member; f.) a child resistant inner locking mechanism is connected to at least one of the central member and the firing cap that is unlockable and, when locked, prevents movement of the firing cap toward the distal end of the central member, and permits movement of the firing cap toward the distal end of the central member when the locking member is unlocked; and, g.) a plurality of dosage choice stops extending from the central member so that each stop may intersect a bar attached to the spring when the spring is decompressed and a corresponding mono dose indicator is selected, the stops being different lengths adapted to meter a different predefined monodose and to stop movement of the spring such that when spring movement is stopped by a longer length stop the piston penetrates into a shorter distance within the monodose package thereby providing for less medicine to be dispensed when the monodose medicine package is punctured and when the spring movement is stopped by a shorter length stop the piston penetrates into a longer distance within the monodose package thereby providing for more the medicine to be dispensed when the monodose medicine package is punctured; wherein the force is required to overcome the frictional force between the high friction engaging surface of the piston and the piston shaft is greater than the force required to compress the spring; and, wherein a user may procure a monodose of medicine with the device when loaded with a monodose medicine package by (i.) removing the inner locking mechanism from its locked position to its unlocked position; (ii) moving the inner locking mechanism from its locked position to its unlocked position; (iii) placing fingers in front of the finger support means and applying compressive force against the firing cap to move the firing cap towards the central member so as to compress the spring and to move the firing cap closer toward the central member to push the piston out of the piston engaging friction area of the shaft so as to sufficiently reduce friction such that the spring automatically and with significant celerity, fires the piston and causes the distal end of the piston to puncture the monodose medicine package and to drive medicine therefrom to and through the spray nozzle in a manner that is user independent as to dosage amount, once selected, and dosage speed of delivery to yield a consistent, predetermined profile spray.

In another preferred embodiment, a monodose nasal sprayer device comprises: a.) an elongated main housing having a distal end adapted for partial insertion into one of a human nasal cavity and an animal nasal cavity and having a proximal end adapted to receive and hold a single use monodose medicine package-supporting central member, the elongated main housing having a spray discharge nozzle located at its distal end, the elongated main housing has medicine-receiving chamber connected to the nozzle and biased toward the single use monodose medicine package-supporting central member; b.) the single use medicine package-supporting central member is connected to the proximal end of the elongated main housing, the central member has a distal end adapted to support a single use monodose medicine package so as to be positioned at the chamber, and having a piston shaft with a piston-engaging high friction area end and having a piston-releasing area distal end; c.) a proximal end plug cap piston is located in the piston shaft, the proximal end plug cap piston has a medicine container breaking distal end plug cap piston, has a central area with a high friction engaging surface to engage the proximal end of the piston shaft and has an extended proximal end functionally connected to a firing cap; d.) a firing cap is connected to the proximal end of the central member and is moveable toward the distal end of the central member by compressive force, the firing cap having a piston-receiving cavity and a drive spring located in the cavity, the extended proximal end of the piston is functionally connected to the spring; e.) finger support means is located on the outside of one of the main housing and the central member; f.) a child resistant inner locking mechanism is connected to at least one of the central member and the firing cap that is unlockable and, when locked, prevents movement of the firing cap toward the distal end of the central member, and permits movement of the firing cap toward the distal end of the central member when the locking member is unlocked; and g.) the single use monodose medicine package including the proximal end plug cap piston and the distal end plug cap piston wherein when the firing cap is fired and the spring pushes the proximal end plug cap piston into the distal end plug cap piston thereby driving the medicine through the spray nozzle; wherein the force is required to overcome the frictional force between the high friction engaging surface of the proximal end plug cap piston and the piston shaft is greater than the force required to compress the spring; and, wherein a user may procure a monodose of medicine with the device when is loaded with a single use monodose medicine package by (i.) removing the inner locking mechanism from its locked position to its unlocked position; (ii) moving the inner locing mechanism from its locked position to its unlocked; (iii) placing fingers in front of the finger support means and applying compressive force against the firing cap to move the firing cap towards the central member so as to compress the spring and to move the firing cap closer toward the central member to push the proximal end plug cap piston out of the piston engaging friction area of the shaft so as to sufficiently reduce friction such that the spring automatically and with significant celerity, fires the piston and causes the distal end of the piston to force the proximal end plug cap to move as a piston through the single use monodose medicine package and to force the distal end plug cap to be removed from the plug and to move as a piston thereby driving the medicine therefrom to and through the spray nozzle in a manner that is user independent as to dosage amount and dosage speed of delivery to yield a consistent, predetermined profile spray.

Additional features, advantages, and embodiments of the invention may be set forth or apparent from consideration of the following detailed description, drawings, and claims. Moreover, it is to be understood that both the foregoing summary of the invention and the following detailed description are exemplary and intended to provide further explanation without limiting the scope of the invention as claimed.

### BRIEF DESCRIPTION OF THE DRAWINGS

The accompanying drawings, which are included to provide a further understanding of the invention and are incorporated in and constitute a part of this specification, illustrate preferred embodiments of the invention and together with the detail description serve to explain the principles of the invention. In the drawings:

FIG. 1 illustrates a front view of one preferred embodiment of a present invention manually operated monodose nasal sprayer having a child resistant outer locking mechanism;

FIG. 2 illustrates a front cut view of one preferred embodiment of a present invention monodose nasal sprayer having a child resistant outer locking mechanism;

FIG. 3 illustrates a front cut view of the present invention of FIG. 2 being compressed by a user's fingers to load the spring;

FIGS. 4 illustrates a front cut view of the present invention of FIG. 2 in the firing position administering a metered dose;

FIG. 5 illustrates an exploded side cut view of the central member and firing cap of FIG. 2;

FIG. 6 illustrates an exploded side view of the central member, firing cap, main housing, piston shaft and piston of FIG. 2; and

FIGS. 7, 8 and 10 show top, front and bottom views of yet another embodiment of a present invention monodose nasal sprayer.

### INCORPORATION BY REFERENCE

The entire application known as U.S. patent application Ser. No. 12/011,117 filed on Jan. 24, 2008, entitled “Manually Operated Monodose Nasal Sprayer” by the same inventor herein, is hereby incorporated into this present application by reference.

### DETAILED DESCRIPTION OF THE EMBODIMENTS

Referring now to the drawings, like reference numerals designate corresponding parts throughout the several views.

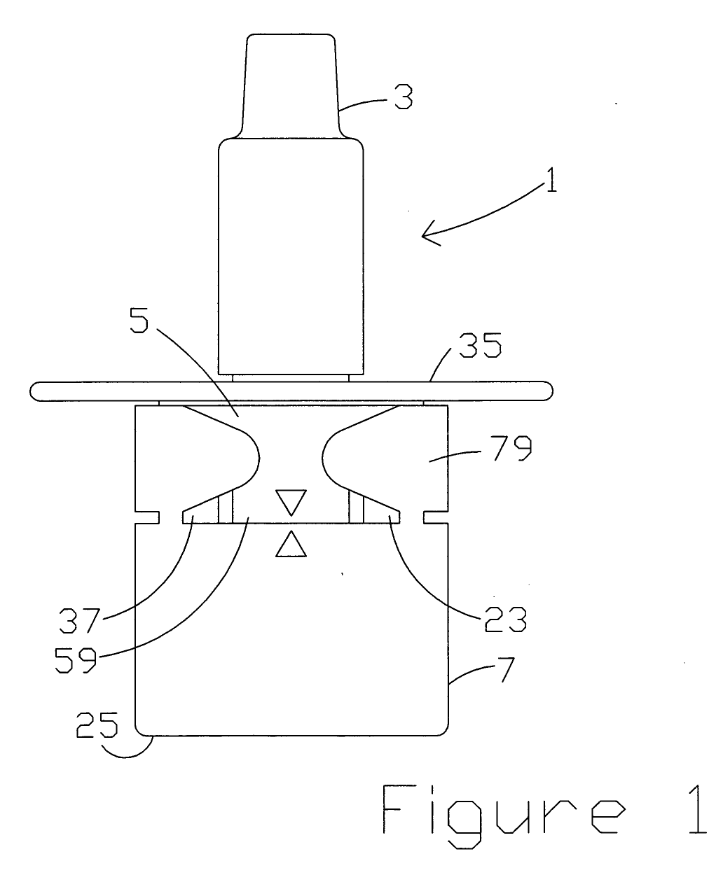

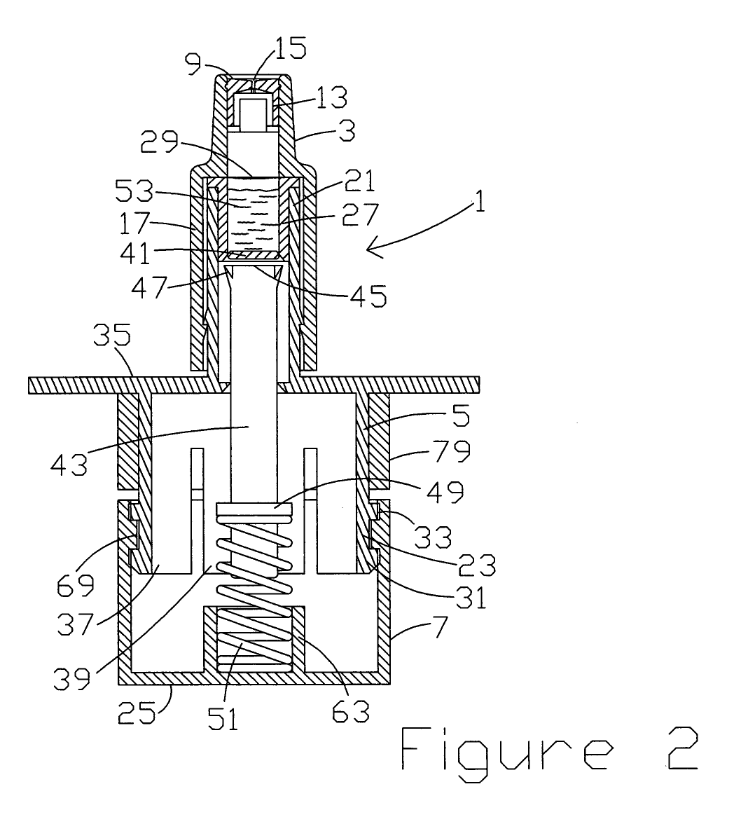

FIG. 1 illustrates a front view of one preferred embodiment of a present invention monodose nasal sprayer 1 having a child resistant outer locking mechanism 79 while FIG. 2 illustrates a front cut view of one preferred embodiment of a present invention monodose nasal sprayer 1 having a child resistant outer locking mechanism 79. It includes an elongated main housing 3 having a distal end 9 adapted for partial insertion into one of a human nasal cavity and an animal nasal cavity. Main housing 3 also has a proximal end adapted to receive and hold a single-use monodose medicine package-supporting central member 5. The elongated main housing 3 has a spray discharge mechanism 13 and nozzle 15 located at its distal end 9. It also has medicine-receiving chamber 17 connected to the nozzle 15 via spray discharge mechanism 13. Chamber 17 is biased toward the single-use monodose medicine package-supporting central member 5 and is adjacent to central member 5's medicine package support, as shown.

The single-use medicine package-supporting central member 5 is connected to the proximal end of the elongated main housing 3. The central member 5 has a distal end 21 adapted to support monodose medicine package 53 so as to be positioned at (directly below) main housing chamber 17, as shown. Monodose medicine package 53 has a rupturable top 29 and a rupturable push-in bottom 41. Central member 5 also has a piston shaft 27 with a piston-releasing area distal end 21. There is a piston 47 located in the piston shaft 27, with piston 47 having a medicine container breaking distal end 45, having a central area 43 to engage the extended proximal end with flange 49, functionally connected to firing cap 7. The piston shaft 27 has a piston-supporting area open at the distal end 45 and adapted to extend piston 47 therein.

Firing cap 7 is connected to the proximal end 23, 37, 39 and 59 of the central member 5 and is moveable toward the distal end 21 of central member 5 by compressive force. The firing cap 7 has a piston-receiving cavity 63 and a drive spring 51 located in the cavity 63. The extended proximal end with flange 49 of the piston 47 is functionally connected to the spring 51 as shown.

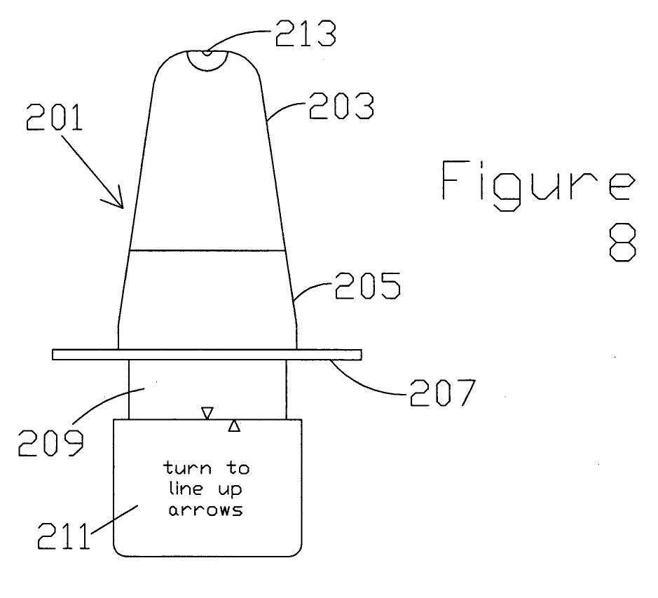

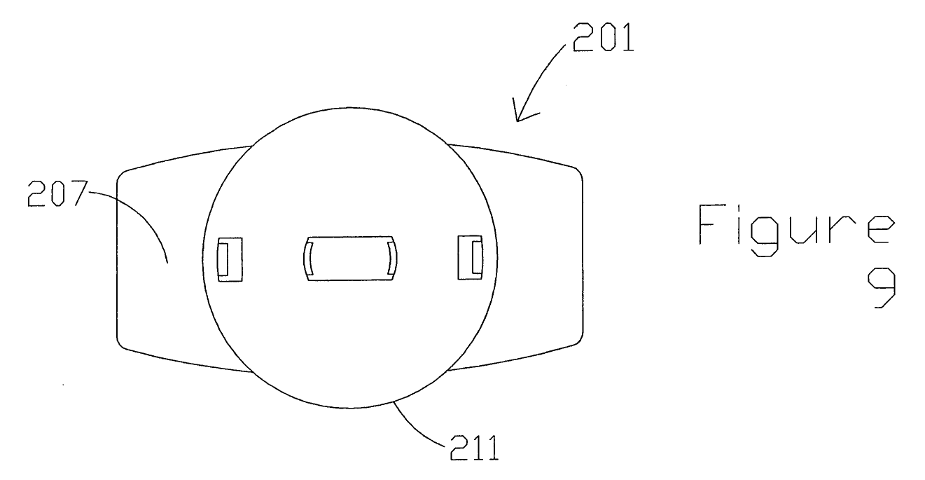

There is a finger support means 35 located on the outside of one of the main housing 3 and the central member 5. In this drawing, it is located on the outside of central member 5, but would be equally functional if located on the outside of main housing 3. A child resistant inner locking mechanism is connected to at least one of the central member 5 and the firing cap 7 that is unlockable and, when locked, prevents upward movement of the firing cap 7 toward the proximal end 23, 37, 39 and 59 of the central member 5, and permits upward movement of the proximal end 25 of the firing cap 7 toward the proximal end 23, 37, 39 and 59 of the central member 5 when the locking member is unlocked. In this drawing the child resistant locking mechanism is hidden, but is visible as shown in FIG. 8.

A post firing lock-up mechanism is connected to the central member 5 and the firing cap 7 such that, once the device has been fired and the firing cap 7 has been advanced forward (upward) toward the central member 5, this locking mechanism is activated and locks up so as to prevent movement of the firing cap 7 away from the proximal end 23, 37, 39 and 59 of the central member 5. In this embodiment, proximal end is divided into four sections, with right section 23, left section 37, back section 39 and front section 59. This allows the proximal ends 23, 37, 39 and 59 to compressively fit into firing cap 7, securing central member 5 in place. The proximal end 23 of central member 5 has two external protrusions 31 and 33 and firing cap 7 has an internal protrusion 69. (For purposes of this drawing, the protrusions are 360 degree full circle protrusions, but could be segmented or opposing pairs, or one component could be full circle and the other be segmented, e.g., protrusion 69 is full circle and protrusions 31 and 33 are arcs.) A recess 55 is located above the internal protrusion 69 on the firing cap 7 for receiving external protrusion 31 of the central member 5. The inner portion 39 of the proximal end 23 of the central member 5 surrounds the central area 43 of piston 47 and piston proximal end with flange 49 during use.

The child resistant outer locking mechanism 79 is connected to the firing cap 7 and the central member 5 and is adapted to prevent movement of the firing cap 7. The outer locking mechanism 79 is a tear away system which removably connects the firing cap 7 to the central member 5.

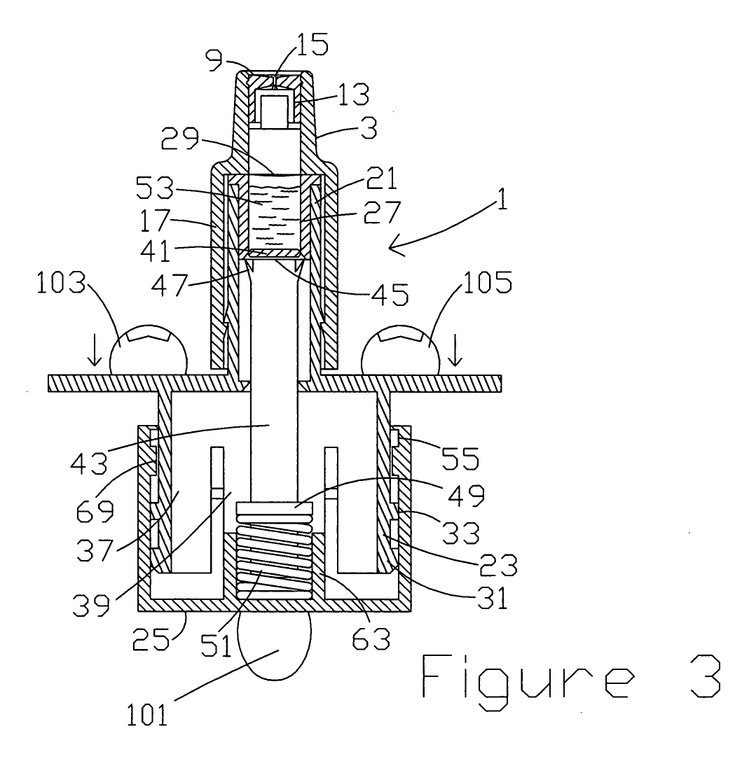

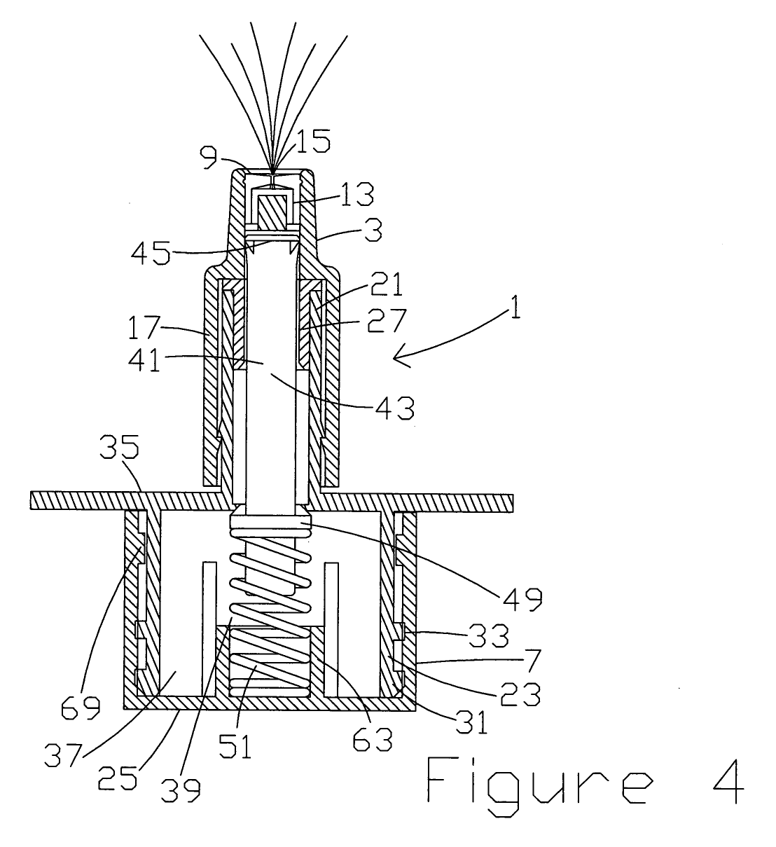

FIG. 3 illustrates a front cut view of the present invention of FIG. 2 being compressed by a user's fingers 101, 103, 105 to load the spring 51 for firing. FIGS. 4 illustrates a front cut view of the present invention of FIG. 2 in the firing position administering a metered dose as described below. A user may procure a monodose of medicine with the device when loaded with a breakable monodose medicine package 51, in this case with rupturable top 29 and rupturable push-in bottom 41. The user accomplishes the results by (i) removing the outer locking mechanism 79 so that its position is unlocked; (ii) moving the inner locking mechanism from its locked position to its unlocked position; (iii) placing fingers 103 and 105 in front of the finger support means and a finger 101 on the proximal end 25 of the firing cap 7 for support and applying compressive force against the firing cap 7 to move the firing cap 7 towards the central member 5 so as to compress the spring 51 and to move the firing cap 7 closer toward the central member 5 to push piston 47 so as to puncture the rupturable push-in bottom 41 of single use breakable monodose medicine package 53 that the spring 51 automatically and with significant celerity, fires the piston 47 and causes the distal end 45 of the piston to drive medicine therefrom to and through the spray nozzle 15 in a manner that is user independent as to dosage amount and dosage speed of delivery to yield a consistent, predetermined profile spray. The post firing lock-up mechanism 31, 33 and 69 automatically locks the device 1 after use so that it cannot be refired.

The piston 47 is able to puncture rupturable push-in bottom 41 of single use breakable monodose medicine package 53 because the height of piston 47 is greater than the sum of the heights of the central member 5 and firing cap 7. The force required to puncture rupturable push-in bottom 41 is greater than the force required to compress spring 51. This relationship between the forces required to operate the device ensure that the spring compresses fully without damaging the breakable monodose medicine package 53. Once the spring 51 is fully compressed, additional compression of the monodose nasal sprayer 1 forces piston 47 towards the monodose medicine package 53, puncturing the rupturable push-in bottom 41 and delivering a measured dosage as described above.

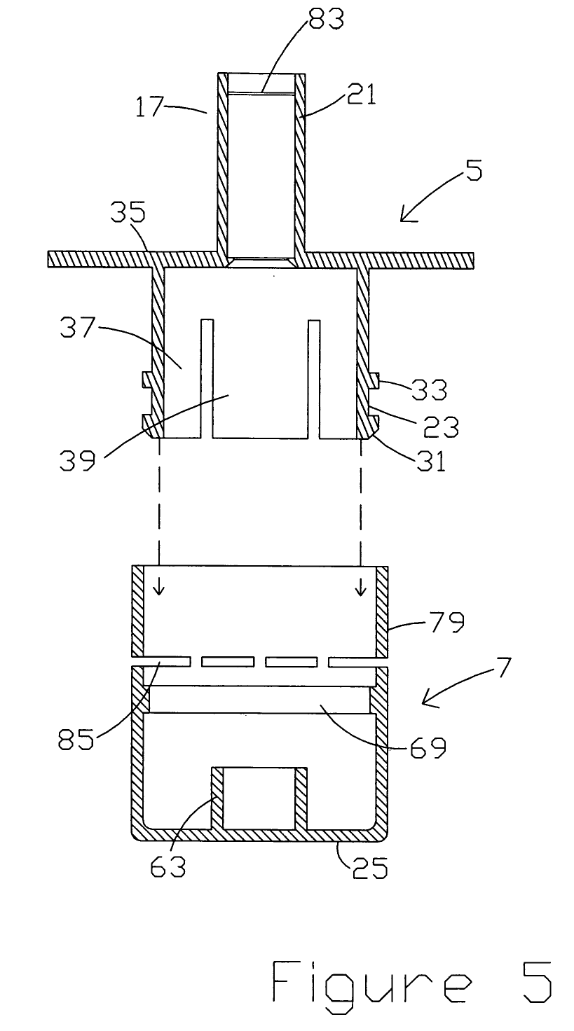

FIG. 5 illustrates an exploded side cut view of the central member and firing cap of FIG. 2. Central member 5 is constructed as described above, with main housing chamber 17, distal end 21, finger support means 35, outer portion 37 of the proximal end 23 of central member 5 has two external protrusions 31 and 33 and inner portion 39 of the proximal end 23 of central member 5. The piston-supporting area 83 is clearly visible in FIG. 5 with piston 47 removed. Central member 5 is adapted to connect to firing cap 7 as described above. Firing cap 7 is composed of proximal end 25, piston-receiving cavity 63, internal protrusion 69, and child resistant outer locking mechanism 79. In the present embodiment, internal protrusion 69 is a 360 degree full circle protrusion for connecting to the two external protrusions 31 and 33 of the central member 5. Child resistant outer locking mechanism 79 is connected to the firing cap 7 by perforations 85.

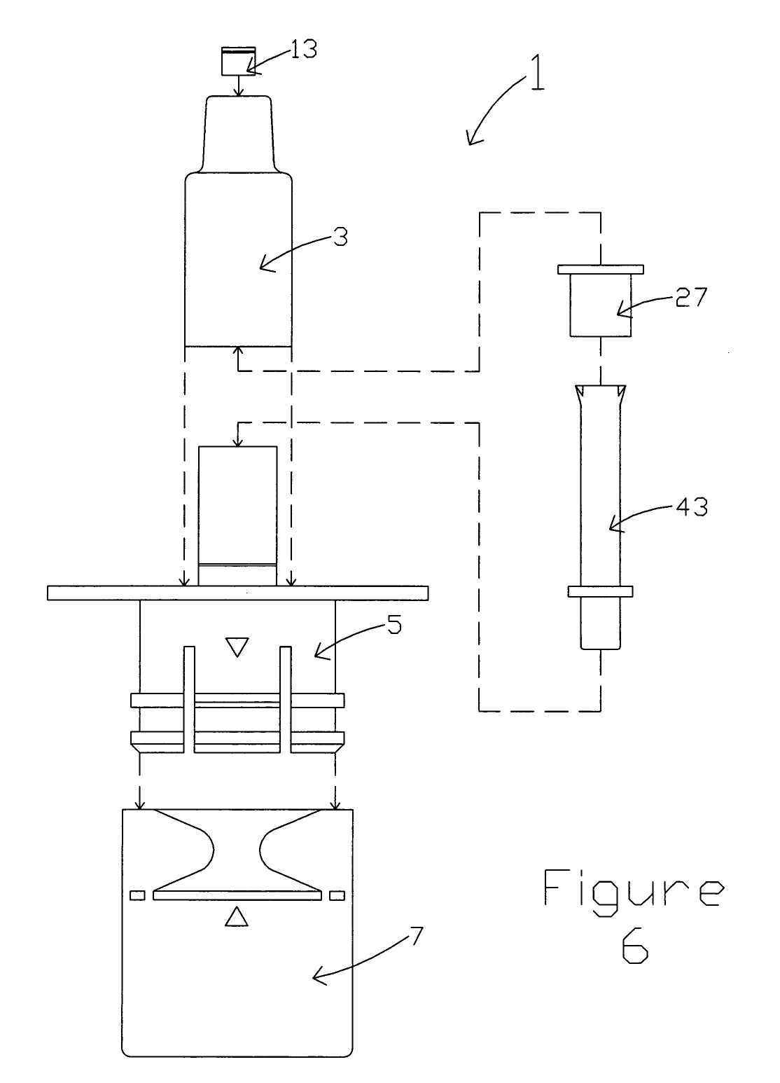

FIG. 6 illustrates an exploded side view of the central member 5, firing cap 7, main housing 3, piston shaft 27 and piston central area 43 as previously described above in FIG. 2. The position of each member relative to one another is well illustrated by FIG. 6. Discharge mechanism 13 is located at the distal end 9 of main housing 3. The piston shaft 27 is located within main housing 3, and piston central area 43 is partially located in the piston shaft 27. Main housing 3 is connected to central member 5 through a connection means such as corresponding screw components or corresponding snap components. Central member 5 connects to firing cap 7 as described above.

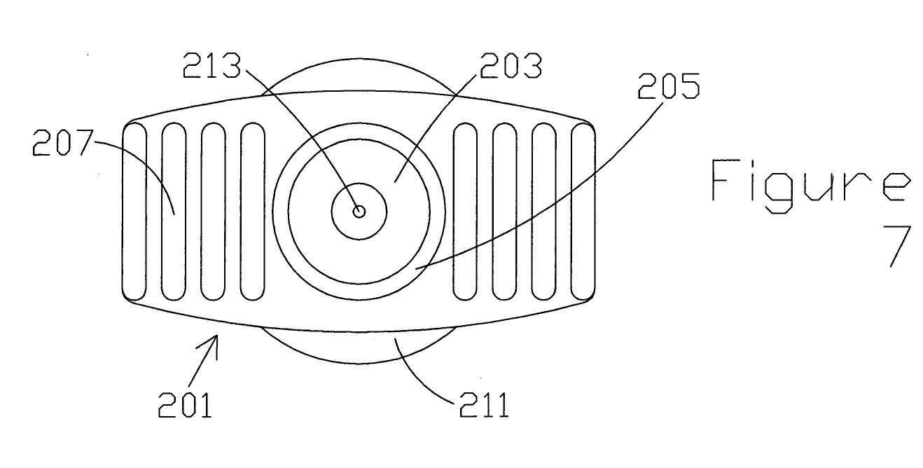

FIGS. 7, 8 and 9 illustrate top, front and bottom views of yet another embodiment of a present invention monodose nasal sprayer 201. All of these Figures have like numbered components and the Figures are discussed collectively.

Sprayer 201 includes an elongated main housing 203 having a distal end adapted for partial insertion into one of a human nasal cavity and an animal nasal cavity. Main housing 203 also has a proximal end adapted to receive and hold a breakable monodose medicine package-supporting central member 205. In this embodiment, the connection between the main housing 203 and the central member 205 is a force fit, as shown. The elongated main housing 203 has a spray discharge mechanism nozzle 213 located at its distal end.

Firing cap 211 is connected to the proximal end of the central member 205 and is moveable toward the distal end of central member 205 by compressive force. This compressive force is applied by finger support means 207 located on the outside of central member 205.

A child resistant inner locking mechanism 209 clearly visible in FIG. 8. Child resistant inner locking mechanism 209 must be twisted so that the arrows on the child resistant inner locking mechanism 209 and firing cap 211 line up in order to use the sprayer 201. When locked, this prevents upward movement of the firing cap 211 toward the central member 205, and permits upward movement of the firing cap 211 toward the central member 205 when the locking member is unlocked.

The child resistant locking mechanism also performs as a post firing lock-up mechanism such that, once the device has been fired and the firing cap 211 has been advanced forward (upward) toward the central member 205, this locking mechanism is activated and locks up so as to prevent movement of the firing cap 211 away from the central member 205.

Although particular embodiments of the invention have been described in detail herein with reference to the accompanying drawings, it is to be understood that the invention is not limited to those particular embodiments, and that various changes and modifications may be effected therein by one skilled in the art without departing from the scope or spirit of the invention as defined in the appended claims.

What is claimed is:

1. A manually operated monodose nasal sprayer device, which comprises:
a) an elongated main housing having a distal end adapted for partial insertion into one of a human nasal cavity and an animal nasal cavity and having a proximal end adapted to receive and hold a single use monodose medicine package-supporting central member, said elongated main housing having a spray discharge nozzle located at its distal end, said elongated main housing having medicine-receiving chamber connected to said nozzle and biased toward said single use monodose medicine package-supporting central member;
b) said single use medicine package-supporting central member being connected to said proximal end of said elongated main housing, said central member having a distal end adapted to support a single use monodose medicine package so as to be positioned at said chamber of said elongated main housing, said single use monodose medicine package-supporting central member having a piston shaft having a piston-supporting area open at said distal end and adapted to extend a piston therein;
c) said piston being partially located in said piston shaft, said piston having a medicine container breaking distal end, and having a downwardly extended proximal end functionally connected to a firing cap;
d) said firing cap connected to said proximal end of said central member and being moveable toward said distal end of said central member by compressive force, said firing cap having a piston-receiving cavity and a drive spring located in said cavity, said extended proximal end of said piston being functionally connected to said spring;
e) finger support means located on the outside of one of said main housing and said central member;
f) a child resistant inner locking mechanism connected to at least one of said central member and said firing cap that is unlockable and, when locked, prevents movement of said firing cap toward said distal end of said central member, and permits movement of said firing cap toward said distal end of said central member when said locking member is unlocked; and,
g) a child resistant outer locking mechanism connected to said firing cap and said central member, adapted to prevent movement of said firing cap, wherein said child resistant outer locking mechanism is a tear away system;
wherein said piston has a predetermined height, said central member piston-supporting area having a predetermined height, and said firing cap has a predetermined height, and wherein the height of said piston is greater than the sum of the height of said central member piston-supporting area and said firing cap; and,
wherein a user may procure a monodose of medicine with said device when loaded with a single use monodose medicine package by (i) removing said outer locking mechanism so that its position is unlocked; (ii) moving said inner locking mechanism from its locked position to its unlocked position; and (iii) placing fingers in front of the finger support means and applying compressive force against said firing cap to move said firing cap towards said central member so as to compress said spring and to move said firing cap closer toward said central member to push said piston past said piston-supporting area of said shaft so as to puncture said single use monodose medicine package such that said spring then automatically and with significant celerity, fires said piston and causes said distal end of said piston to drive medicine therefrom to and through said spray nozzle in a manner that is user independent as to dosage amount and dosage speed of delivery to yield a consistent, predetermined profile spray.

2. The manually operated monodose nasal sprayer device of claim 1 which further includes a single use monodose medicine package positioned in said central member, said single use monodose medicine package having side walls, a rupturable top and a rupturable push-in bottom.

3. The manually operated monodose nasal sprayer device of claim 1 wherein said piston shaft and said piston have corresponding circular peripheries from a top view.

4. The manually operated monodose nasal sprayer device of claim 1 wherein said distal end of said central member is connected to said proximal end of said main housing through connection means wherein said connection means is selected from one of the group consisting of corresponding screw components and corresponding snap components.

5. The manually operated monodose nasal sprayer device of claim 4 wherein said screw components include a one way locking thread stop so that said distal end of said central member and said proximal end of said main housing cannot be unscrewed once screwed together.

6. The manually operated monodose nasal sprayer device of claim 1 wherein said proximal end of said piston includes a flange that fits atop said spring that fits into said firing cap cavity.

7. The manually operated monodose nasal sprayer device of claim 3 wherein said outer locking mechanism connection means includes a perforated tab surrounding a circumference of at least one of said firing cap and said central member.

8. A manually operated monodose nasal sprayer device, which comprises:
a) an elongated main housing having a distal end adapted for partial insertion into a one of human nasal cavity and an animal nasal cavity and having a proximal end adapted to receive and hold a monodose medicine package-supporting central member, said elongated main housing having a spray discharge nozzle located at its distal end, said elongated main housing having medicine-receiving chamber connected to said nozzle and biased toward said monodose medicine package-supporting central member;
b) said single use medicine package-supporting central member being connected to said proximal end of said elongated main housing, said central member having a distal end adapted to support a single use monodose medicine package so as to be positioned at said chamber of said elongated main housing, said single use monodose medicine package-supporting central member having a piston shaft having a piston-supporting area open at said distal end and adopted to extend a piston therein;
c) said piston being partially located in said piston shaft, said piston having a medicine container breaking distal end, and having a downwardly extended proximal end functionally connected to a firing cap;
d) said firing cap connected to said proximal end of said central member and being moveable toward said distal end of said central member by compressive force, said firing cap having a piston-receiving cavity and a drive spring located in said cavity, said extended proximal end of said piston being functionally connected to said spring;
e) finger support means located on the outside of one of said main housing and said central member;
f) a child resistant inner locking mechanism connected to at least one of said central member and said firing cap that is =lockable and, when locked, prevents movement of said firing cap toward said distal end of said central member, and permits movement of said firing cap toward said distal end of said central member when said locking member is unlockled; and,
g) a plurality of dosage choice stops extending from said central member so that each stop may intersect a bar attached to said spring when said spring is decompressed and a corresponding monodose indicator is selected, said stops being different lengths adapted to meter a different predefined monodose and to stop movement of said spring such that when spring movement is stopped by a longer length stop said piston penetrates into a shorter distance within said monodose package thereby providing for less medicine to be dispensed when said monodose medicine package is punctured and when said spring movement is stopped by a shorter length stop said piston penetrates into a longer distance within said monodose package thereby providing for more said medicine to be dispensed when said monodose medicine package is punctured;
wherein said piston has a predetermined height, said central member piston-supporting area having a predetermined height, and said firing cap has a predetermined height, and wherein the height of said piston is greater than the sum of the height of said central member piston-supporting area and said firing cap; and,
wherein a user may procure a monodose of medicine with said device when loaded with a single use monodose medicine package by (i) removing said outer locking mechanism so that its position is unlocked; (ii) moving said inner locking mechanism from its locked position to its unlocked position; and (iii) placing fingers in front of the finger support means and applying compressive force against said firing cap to move said firing cap towards said central member so as to compress said spring and to move said firing cap closer toward said central member to push said piston past said piston-supporting area of said shaft so as to puncture said single use monodose medicine package such that said spring then automatically and with significant celerity, fires said piston and causes said distal end of said piston to drive medicine therefrom to and through said spray nozzle in a manner that is user independent as to dosage amount and dosage speed of delivery to yield a consistent, predetermined profile spray.

9. The manually operated monodose nasal sprayer of claim 8 which includes a single use monodose medicine package positioned in said central member, said single use monodose medicine package having side walls, a rupturable top and a rupturable push-in bottom.

10. The manually operated monodose nasal sprayer device of claim 8 wherein said piston shaft and said piston have corresponding circular peripheries from a top view.

11. The manually operated monodose nasal sprayer device of claim 8 wherein said distal end of said central member is connected to said proximal end of said main housing through connection means wherein said connection means is selected from one of the group consisting of corresponding screw components and corresponding snap components.

12. The manually operated monodose nasal sprayer device of claim 11 wherein said screw components include a one way locking thread stop so that said distal end of said central member and said proximal end of said main housing cannot be unscrewed once screwed together.

13. The manually operated monodose nasal sprayer device of claim 8 wherein said proximal end of said piston includes a flange that fits atop said spring that fits into said firing cap cavity.

14. The manually operated monodose nasal sprayer device of claim 10 wherein said inner locking mechanism is a rotating locking mechanism with a first position being a lock position to inhibit advancement of said firing cap and a second position being an unlock position to permit advancement of said firing cap, said second position being located at an arc of a predetermined distance from said first position.

15. A manually operated monodose nasal sprayer device, which comprises:
a) an elongated main housing having a distal end adapted for partial insertion into one of a human nasal cavity and an animal nasal cavity and having a proximal end adapted to receive and hold a single use monodose medicine package-supporting central member, said elongated main housing having a spray discharge nozzle located at its distal end, said elongated main housing having medicine-receiving chamber connected to said nozzle and biased toward said single use monodose medicine package-supporting central member;
b) said single use medicine package-supporting central member being connected to said proximal end of said elongated main housing, said central member having a distal end adapted to support a single use monodose medicine package so as to be positioned at said chamber of said elongated main housing, said single use monodose medicine package-supporting central member having a piston shaft having a piston-supporting area open at said distal end and adapted to extend a piston therein;
c) said piston being partially located in said piston shaft, said piston having a medicine container breaking distal end, and having a downwardly extended proximal end functionally connected to a firing cap;
d) said firing cap connected to said proximal end of said central member and being moveable toward said distal end of said central member by compressive force, said firing cap having a piston-receiving cavity and a drive spring located in said cavity, said extended proximal end of said piston being functionally connected to said spring;
e) finger support means located on the outside of one of said main housing and said central member;
f) a child resistant inner locking mechanism connected to at least one of said central member and said firing cap that is unlockable and, when locked, prevents movement of said firing cap toward said distal end of said central member, and permits movement of said firing cap toward said distal end of said central member when said locking member is unlocked; and,
g) said single use monodose medicine package including said proximal end plug cap piston and said distal end plug cap piston wherein when said firing cap is fired and said spring pushes said proximal end plug cap piston into said distal end plug cap piston thereby driving said medicine through said spray nozzle;
wherein said piston has a predetermined height, said central member piston-supporting area having a predetermined height, and said firing cap has a predetermined height, and wherein the height of said piston is greater than the sum of the height of said central member piston-supporting area and said firing cap; and,
wherein a user may procure a monodose of medicine with said device when loaded with a single use monodose medicine package by (i) removing said outer locking mechanism so that its position is unlocked; (ii) moving said inner locking mechanism from its locked position to its unlocked position; and (iii) placing fingers in front of the finger support means and applying compressive force against said firing cap to move said firing cap towards said central member so as to compress said spring and to move said firing cap closer toward said central member to push said piston past said piston-supporting area of said shaft so as to puncture said single use monodose medicine package such that said spring then automatically and with significant celerity, fires said piston and causes said distal end of said piston to drive medicine therefrom to and through said spray nozzle in a manner that is user independent as to dosage amount and dosage speed of delivery to yield a consistent, predetermined profile spray.

16. The manually operated monodose nasal sprayer device of claim 15 wherein said piston shaft and said piston have corresponding circular peripheries from a top view.

17. The manually operated monodose nasal sprayer device of claim 15 wherein said distal end of said central member is connected to said proximal end of said main housing through connection means wherein said connection means is selected from one of the group consisting of corresponding screw components and corresponding snap components.

18. The manually operated monodose nasal sprayer device of claim 17 wherein said screw components include a one way locking thread stop so that said distal end of said central member and said proximal end of said main housing cannot be unscrewed once screwed together.

19. The manually operated monodose nasal sprayer device of claim 15 wherein said proximal end of said piston includes a flange that fits atop said spring that fits into said firing cap cavity.

20. The manually operated monodose nasal sprayer device of claim 16 wherein said inner locking mechanism is a rotating locking mechanism with a first position being a lock position to inhibit advancement of said firing cap and a second position being an unlock position to permit advancement of said firing cap, said second position being located at an arc of a predetermined distance from said first position.
